(19) United States
(12) Patent Application Publication
(10) Pub. No.: US 2021/0214760 A1
(43) Pub. Date: Jul. 15, 2021

(54) SYSTEM AND METHOD FOR MANUFACTURING ERROR MITIGATED POLYNUCLEOTIDES

(71) Applicant: APDN (B.V.I.) Inc., Tortola (VG)

(72) Inventors: Stephen HUGHES, Port Jefferson, NY (US); James A. Hayward, Stony Brook, NY (US); Brian Viscount, Mt. Sinai, NY (US)

(21) Appl. No.: 17/144,250

(22) Filed: Jan. 8, 2021

Related U.S. Application Data
(60) Provisional application No. 62/958,829, filed on Jan. 9, 2020.

(51) Int. Cl. C12P 19/34

(52) U.S. Cl. CPC C12P 19/34

(57) ABSTRACT

The present invention relates generally to molecular biology, and more specifically, to a system and method for the manufacture of quantities of error mitigated polynucleotides via the polymerase chain reaction (PCR).

### CROSS-REFERENCE TO RELATED APPLICATION

This application claims the benefit of U.S. Provisional Patent Application No. 62/958,829, filed on Jan. 9, 2020, which is hereby incorporated by reference in its entirety.

### TECHNICAL FIELD

The present invention relates generally to molecular biology, and more specifically, to a system and method for the manufacture of quantities of error mitigated polynucleotides via the polymerase chain reaction (PCR).

### BACKGROUND OF THE INVENTION

With the rise of synthetic biology, the era of creating new functional genes, genetic networks and whole genomes is upon us. A necessity for synthetic biology, and its therapeutic offshoots such as adoptive cell therapy, gene therapy, RNA based therapies and nucleic acid-based vaccines, is the production of large quantities of polynucleotides with a desired nucleotide sequence.

Currently, large quantities of polynucleotides are produced via bacterial plasmid propagation, and more recently, enzymatic processes such as the polymerase chain reaction (PCR). While enzymatic amplification technologies hold great promise to produce large quantities of polynucleotides in a rapid and cost effective manner, the resulting polynucleotides unavoidably contain sequence errors such as deletions, insertions or base substitutions which must be mitigated if the resultant polynucleotides will be used in therapeutic or prophylactic settings. In addition, in enzymatic amplification systems, purification of the amplified polynucleotides with the correct nucleotide sequence is challenging often due to the small size of the target polynucleotides and the current limitations of purification technologies and related analytical chemistry techniques.

Unlike current methods and systems for enzymatic based polynucleotide amplification, nature has evolved sophisticated error-correction mechanisms to ensure that DNA replication proceeds with high fidelity. The known error rates in prokaryotic and eukaryotic replication machineries range from approximately 10⁻⁷ to 10⁻⁸ by proofreading and mismatch repair mechanisms (Schofield M J, Hsieh P. DNA mismatch repair: molecular mechanisms and biological function. Annu Rev Microbiol. 2003; 57:579-608) (Li GM. Mechanisms and functions of DNA mismatch repair. Cell Res. 2008; 18:85-98). In contrast, current enzymatic amplification technologies cannot reach nature's levels of high-fidelity of polynucleotide replication without additional methods and systems of error mitigation.

Therefore, there is an unmet need for a method and system wherein the error rate inherent in enzymatic based polynucleotide amplification can be mitigated to a level equal to or less than naturally occurring polynucleotide replication error rates. In addition, there is a further unmet need for said method and system to increase the efficiency of purifying the amplified polynucleotides with the desired nucleotide sequence.

### SUMMARY OF THE INVENTION

The present invention relates to a polymerase chain reaction (PCR) based method and system of manufacturing error mitigated polynucleotides via the exploitation of single-stranded mismatches in dsDNA molecules at the point(s) of sequence variation from the desired sequence. The single-stranded mismatches may be formed, exploited and mitigated in self-assembling 2-D or 3-D DNA structures that enhance error mitigation and aid polynucleotide purification.

In an embodiment, a method of manufacturing a plurality of error mitigated polynucleotides of a desired sequence is disclosed, said method comprising the steps of: (a) Obtaining a quantity of target polynucleotides of a desired sequence; (b) amplifying the target polynucleotides via the polymerase chain reaction to create a first set of double stranded amplicons; (c) denaturing the first set of double stranded amplicons; (d) annealing the denatured amplicons to create a second set of double stranded amplicons that contain areas of single stranded sequence mismatches at the point in the amplicon where the nucleotide sequence differs from the desired sequence; (e) reacting the second set of double stranded amplicons with a single strand specific nuclease to create cuts at the points of single strand sequence mismatches in the second set of double stranded amplicons to remove sequence error; and; (f) reacting the second set of double stranded amplicons containing cuts at the points of single strand sequence mismatches with a polymerase to introduce the desired sequence at the cut point.

The single strand specific nuclease may be any known single strand specific nuclease or may be chosen from a group consisting of Mung Bean endonuclease, T7 endonuclease I, E. coli endonuclease V, CEL endonuclease, S1 endonuclease, P1 endonuclease. The polymerase utilized may be any suitable polymerase known in the art or may be E. coli DNA polymerase I (Pol I) or any polymerase with a with 3′-5′ and/or 5′-3′ proofreading functionality. The polynucleotides of a desired sequence may be any desired sequence and may contain an expression cassette and noncoding nucleotides any sequence. The noncoding nucleotides may have a G-C content of less than 65%.

In an embodiment, a method of producing an antigen specific immune response in a subject is disclosed, said method said method comprising the steps of: (a) choosing one or more desired antigens for expression within a subject; (b) obtaining a quantity of target polynucleotides of a desired sequence, said target polynucleotides of a desired sequence containing one or more expression cassettes for the desired antigen; (b) amplifying the target polynucleotides via the polymerase chain reaction to create a first set of double stranded amplicons; (c) denaturing the first set of double stranded amplicons; (d) annealing the denatured amplicons to create a second set of double stranded amplicons that contain areas of single stranded sequence mismatches at the point in the amplicon where the nucleotide sequence differs from the desired sequence; (e) reacting the second set of double stranded amplicons with a single strand specific nuclease to create cuts at the points of single strand sequence mismatches in the second set of double stranded amplicons to remove sequence error; (f) reacting the second set of double stranded amplicons containing cuts at the points of single strand sequence mismatches with a polymerase to introduce the desired sequence at the cut point thereby create a plurality of double stranded amplicons with the desired sequence; (g) formulating the plurality of double stranded amplicons with the desired sequence into a therapeutic dose; and (h) administering the therapeutic dose of double stranded amplicons to a subject, wherein the subject produces the desired one or more antigens in response to the therapeutic dose of double stranded amplicons.

In another embodiment, a method of manufacturing a plurality of error mitigated polynucleotides of a desired sequence is disclosed, said method comprising the steps of: (a) obtaining a quantity of target polynucleotides of a desired sequence; (b) amplifying the target polynucleotide via the polymerase chain reaction (PCR) to create a first set of double stranded amplicons; (c) assembling the first set of double stranded amplicons with a DNA scaffold via seaming PCR to create a second set of double stranded amplicons comprising the target polynucleotide and scaffold DNA; (d) denaturing the second set of double stranded amplicons; (e) reacting the denatured second set of double stranded DNA amplicons with one or more complementary or partially complementary DNA sequences to form 2-D or 3-D DNA structures wherein the complimentary target polynucleotide sequences are forced to hybridize in the 2-D or 3-D DNA structure to create areas of single stranded sequence mismatches at the point in the target polynucleotide sequence where the sequence differs from the desired sequence; (f) reacting the 2-D or 3-D DNA structures with a single strand specific nuclease to create cuts at the points of single strand sequence mismatches thereby removing the sequence error in the target polynucleotide sequence; (g) reacting the 2-D or 3-D DNA structures containing cuts at the points of single strand sequence mismatches with a polymerase to introduce the desired sequence at the cut point; and (h) optionally, releasing the target polynucleotide comprised of the desired sequence from the 2-D or 3-D DNA structures.

The 2-D or 3-D DNA structures may contain more than one copy of the target polynucleotide, which may contain an expression cassette. The 2-D or 3-D DNA structures may be single or double stranded and may be of any shape or may be of a scaffold shape. The 2-D or 3-D DNA structures may be of any molecular weight but are preferably between 1 million Daltons and 10 million Daltons in molecular weight. The released target polynucleotide comprised of the desired sequence may be used as a template for other polynucleotides, including but not limited to RNA.

In an alternative embodiment, a method of manufacturing a plurality of error mitigated polynucleotides of a desired sequence is disclosed, said method comprising the steps of: (a) constructing an expression vector scaffold polynucleotide comprising an expression vector of a desired sequence assembled to a polynucleotide that includes a 5′cleavable stacking element, scaffold anchor, 75% GC extractor segment, a no-complement spacer, at least a 65% GC scaffold anchor, and a 3′ cleavable stacking element; (b) constructing a right pin mirror scaffold polynucleotide comprising a 5′ cleavable stacking element, a negative strand (5′ to 3′) operational gene (encoding CRISPR/CAS9, Guide RNA, or DNA delivery components), at least a 65% GC scaffold anchor pin foot 5′, a 3′ scaffold anchor short foot, and a 3′ cleavable stacking element; (c) constructing an operational scaffold polynucleotide comprising of a 5′ cleavable stacking element, a positive strand (5′ to 3′) operational gene (encoding CRISPR/CAS9, Guide RNA, or DNA delivery components) gene, at least a 65% GC strong lock pin foot 5′, at least a 75% GC extractor segment slide long lock, a no-complement spacer, a 3′ scaffold anchor short foot, and a 3′ cleavable stacking element; (d) constructing a left pin mirror scaffold polynucleotide comprising an expression vector of a desired sequence assembled to a polynucleotide fragment which includes a 5′ cleavable stacking element, a positive strand (5′ to 3′) operational gene (encoding CRISPR/CAS9, Guide RNA, or DNA delivery components), scaffold anchor short foot 5′, at least a 65% GC strong lock pin 3′, and a 3′ cleavable stacking element, wherein the expression vector scaffold polynucleotide, right pin mirror scaffold polynucleotide, operational scaffold polynucleotide, and left pin mirror scaffold polynucleotide are denatured and annealed in a common reaction vessel to form polynucleotides with self-assembling secondary structures, wherein said secondary structures provide for areas of single stranded sequence mismatches at the point in the expression vector where the nucleotide sequence of the expression vector differences from the desired sequence of the expression vector; (e) reacting the polynucleotides with self-assembling secondary structures with a single strand specific nuclease to create cuts at the points of single strand sequence mismatches in the expression cassettes thereby removing the sequence error; and (f) reacting the polynucleotides with self-assembling secondary structures containing cut at the points of single strand sequence mismatches to a polymerase that can perform nick translation to introduce the desired sequence at the cut point. The self-assembling secondary structures may be of any molecular weight, preferably between 1 million and 100 million Daltons, and more preferably a molecular weight of at least 5 million Daltons.

### BRIEF DESCRIPTION OF THE FIGURES

FIG. 1 is a flow diagram of one embodiment of the method and system of the present invention.

FIG. 2 is a schematic drawing of one embodiment of the system of the present invention.

FIG. 3 is a diagram of exemplary 2-D or 3-D DNA structures incorporating on or more target polynucleotides in one embodiment of the present invention.

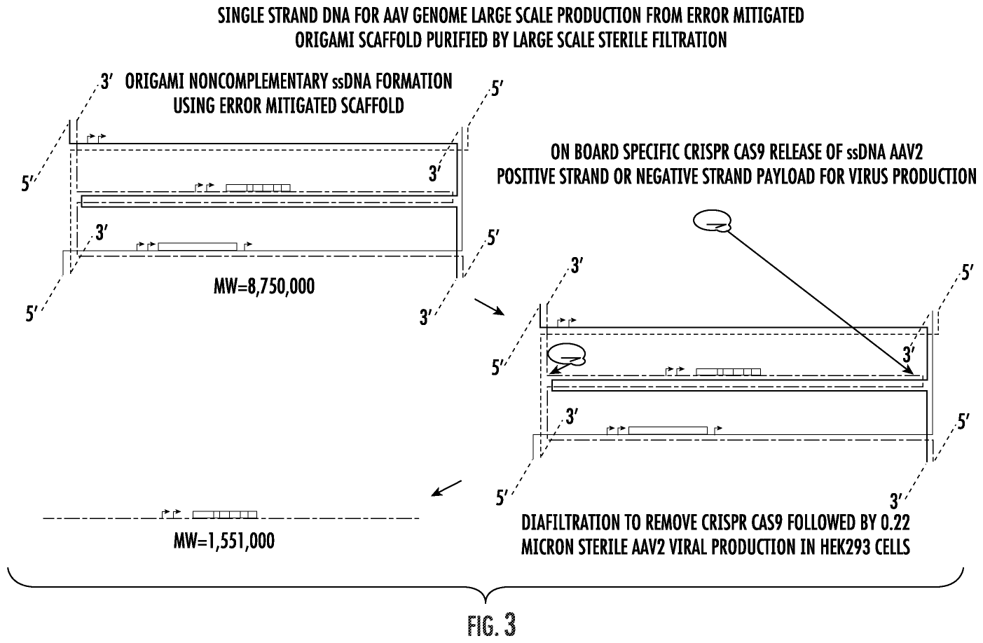

FIG. 4 illustrates a diagram of exemplary 2-D or 3-D DNA structures incorporating one or more target polynucleotides in one embodiment of the present invention.

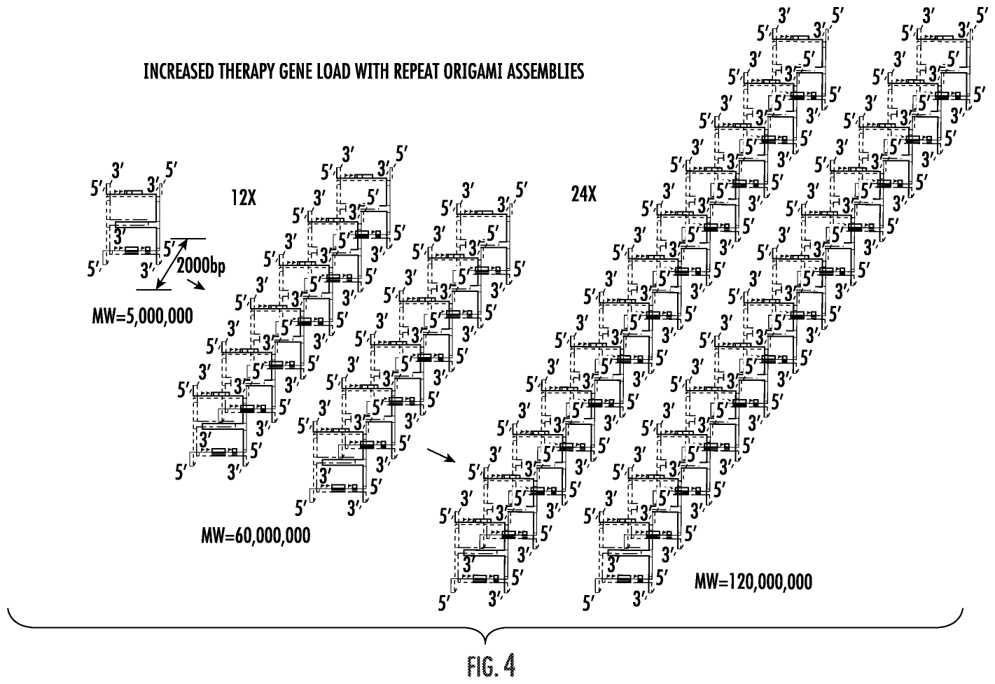

### DETAILED DESCRIPTION OF THE INVENTION

The term “amplicon” as used herein means a piece of DNA or RNA that is the product of an enzymatic or chemical based amplification event or reaction. Amplification events or reactions include, without limitation, the polymerase chain reaction (PCR), loop mediated isothermal amplification, rolling circle amplification, nucleic acid sequence base amplification, and ligase chain reaction or recombinase polymerase amplification. An amplicon may be comprised of single stranded and/or double stranded DNA, and/or a combination thereof.

The term “continuous flow PCR device” means a PCR device as disclosed in U.S. Pat. Nos. 8,293,471, 8,986,982 and 8,163,489 or other PCR device wherein a fluid containing a PCR reaction mixture is transported through a reaction vessel.

The term “expression” refers to the transcription and/or translation of an expression cassette.

The term “expression cassette” means a DNA or other polynucleotide sequence consisting of one or more genes and the sequences controlling their expression. At a minimum, an expression cassette shall include a promoter (or other expression control sequence) and an open reading frame (ORF). As used herein, an expression cassette may be a polynucleotide or a target polynucleotide. An expression cassette may also include additional DNA constructs or elements, which may include, without limitation, expression control sequences and configurations for episomal nuclear persistence and/or episomal nuclear replication. An expression cassette can be single or double stranded. Expression cassettes may also be configured for increase expression via the use of high-level dual promoters, SUMO and ubiquitin expression tags/chaperons in-frame, as well as short effective terminators.

The term “expression control sequence” means a nucleic acid sequence that directs transcription of a nucleic acid and/or open reading frame. An expression control sequence can be a promotor or an enhancer.

The term a “subject” is any mammal, including without limitation humans, monkeys, farm animals, pets, horses, dogs and cats. In an exemplary embodiment, the subject in human.

The term “large-scale PCR” means a PCR reaction wherein the total PCR reaction volume is greater than 0.7 liters. Large-scale PCR may be performed in a single reaction vessel or may be performed in a plurality of reaction vessels simultaneously.

The term “scaffold DNA” means noncoding DNA utilized for the purposes of forming a 2-D or 3-D DNA structure.

The term “staple DNA” or “staple strand” means an oligonucleotide of a complimentary sequence to a region of the scaffold DNA that assists in the formation of a 2-D or 3-D DNA structure.

The use of the alternative (e.g., “or”) should be understood to mean either one, both, or any combination thereof of the alternatives. As used herein, the indefinite articles “a” or “an” should be understood to refer to “one or more” of any recited or enumerated component.

### Method of Manufacturing a Plurality of Error Mitigated Polynucleotides of a Desired Sequence

In one aspect, the invention provides a method of manufacturing a plurality of error mitigated polynucleotides of a desired sequence, said method comprising: (a) obtaining a quantity of target polynucleotides of a desired sequence; (b) amplifying the target polynucleotides via the polymerase chain reaction to create a first set of double stranded amplicons; (c) denaturing the first set of double stranded amplicons; (d) annealing the denatured amplicons to create a second set of double stranded amplicons that contain areas of single stranded sequence mismatches at the point in the amplicon where the nucleotide sequence differs from the desired sequence; (e) reacting the second set of double stranded amplicons with a single strand specific nuclease to create cuts at the points of single strand sequence mismatches in the second set of double stranded amplicons to remove sequence error; and (f) reacting the second set of double stranded amplicons containing cuts at the points of single strand sequence mismatches with a polymerase to introduce the desired sequence at the cut point.

The single strand specific nuclease may be any suitable nuclease known in the art or may be chosen from Mung Bean endonuclease, T7 endonuclease I, E. coli endonuclease V, CEL endonuclease, S1 endonuclease, or P1 endonuclease. The polymerase may be any suitable polymerase known in the art or may be a polymerase with specific 3′-5′ and/or 5′-3′ proofreading functions. The polymerase may be E. coli DNA polymerase I (Pol I). The target polynucleotides of a desired sequence may contain one or more expression cassettes. The target polynucleotides of a desired sequence may be assembled into a larger polynucleotide also containing noncoding nucleotides with a G-C content of at least 65%. The expression cassette may encode any therapeutic protein, including without limitation one or more proteins, antigens, antibodies, chimeric antigen receptors or T cell receptors. In an embodiment, a ligase enzyme may be used in lieu of or in conjunction with a polymerase to complete and error correct the polynucleotide sequence at the point of the single stranded cut made by a single strand specific nuclease.

The target polynucleotides of a desired sequence may be obtained from any source known in the art. The polynucleotides may be assembled via artificial gene synthesis which may include photolithographic means, oligonucleotide synthesis, solid-phase DNA synthesis or any other means of gene synthesis know in the art capable of producing polynucleotides of the necessary length and fidelity. The target polynucleotide may also be derived from a plasmid via methods known in the art, including, but not limited to, molecular cloning or PCR based methods. Target polynucleotides may also be derived from the assembly of various oligonucleotides into larger polynucleotides via assembly apparatuses such as the BioXp™ (SGI-DNA, United States) or seaming PCR.

The amplification of the target polynucleotides of a desired sequence may occur via any PCR or PCR-based process known in the art. Other enzymatic amplification methodologies may also be used. Exemplary enzymatic amplification methodologies include loop mediated isothermal amplification, rolling circle amplification, nucleic acid sequence base amplification, and ligase chain reaction or recombinase polymerase amplification. The PCR reaction may take place in a large-scale PCR system or thermocycler configured for large-scale enzymatic amplification. The PCR reaction may also take place within microfluidic devices or in one or more “lab on a chip” systems. The PCR reaction may take place in a parallel or massively parallel system comprised of a plurality of microfluidic devices or lab on a chip systems. The microfluidic devices may be configured to operate as a plurality of separate reaction vessels or may be configured to run in a continuous flow modality. A continuous flow PCR system not of microfluid design may also be utilized.

In another embodiment, a method of producing an antigen specific immune response in a subject is disclosed, said method said method comprising the steps of: (a) choosing one or more desired antigens for expression within a subject; (b) obtaining a quantity of target polynucleotides of a desired sequence, said target polynucleotides of a desired sequence containing one or more expression cassettes for the desired antigen; (b) amplifying the target polynucleotides via the polymerase chain reaction to create a first set of double stranded amplicons; (c) denaturing the first set of double stranded amplicons; (d) annealing the denatured amplicons to create a second set of double stranded amplicons that contain areas of single stranded sequence mismatches at the point in the amplicon where the nucleotide sequence differs from the desired sequence; (e) reacting the second set of double stranded amplicons with a single strand specific nuclease to create cuts at the points of single strand sequence mismatches in the second set of double stranded amplicons to remove sequence error; (f) reacting the second set of double stranded amplicons containing cuts at the points of single strand sequence mismatches with a polymerase to introduce the desired sequence at the cut point thereby create a plurality of double stranded amplicons with the desired sequence containing expression cassettes for one or more desired antigens; (g) formulating the plurality of double stranded amplicons with the desired sequence containing expression cassettes for one or more desired antigens into a therapeutic dose; and (h) administering the therapeutic dose of double stranded amplicons to a subject, wherein the subject produces the desired one or more antigens in response to the therapeutic dose of double stranded amplicons.

A therapeutic dose may formulated via any know method or formulation. The plurality of double stranded amplicons may be formulated with lipid nanoparticles (LNP) or other compounds to accomplish non-viral transfection of the double stranded amplicons into a subject's cells. The plurality of double stranded amplicons may be formulated as disclosed in United States Patent Application Publication No. US 2020/0224206 A1, published Jul. 16, 2020, the disclosure of which is hereby incorporated by reference in its entirety. Exemplary LNP compositions for the formulation of the plurality of double stranded amplicons of a desired sequence are disclosed in United States Patent Application Publication No. US 2010/0130588 A1, published May 27, 2010 and United States Patent Application Publication No. US 2015/0165039 A1, published Jun. 18, 2015, the disclosure of which is hereby incorporated by reference in their entirety.

The therapeutic dose of double stranded amplicons may be administered via injection, injection with electroporation, electroporation, intravenous delivery or inhalation into a subject's lungs.

The methods of this invention may also be used to produce a plurality of double stranded amplicons of a desired sequence for all other nucleic acid based therapies, including without limitation, adoptive cell therapy, CAR-T cell therapy, TCR therapy, immune-oncology, gene therapy, the production of adeno-associated virus (rAAV), vaccines and the production of RNA, siRNA and/or mRNA.

In another aspect, the invention provides for a method of manufacturing a plurality of error mitigated polynucleotides of a desired sequence, said method comprising: (a) obtaining a quantity of target polynucleotides of a desired sequence; (b) amplifying the target polynucleotides via the polymerase chain reaction (PCR) to create a first set of double stranded amplicons; (c) assembling the first set of double stranded amplicons with a DNA scaffold via seaming PCR to create a second set of double stranded amplicons comprising the target polynucleotide and scaffold DNA; (d) denaturing the second set of double stranded amplicons; (e) reacting the denatured second set of double stranded DNA amplicons with one or more complementary or partially complementary DNA sequences to form 2-D or 3-D DNA structures wherein the complimentary target polynucleotide sequences are forced to hybridize in the 2-D or 3-D DNA structure to create areas of single stranded sequence mismatches at the point in the target polynucleotide sequence where the sequence differs from the desired sequence; (f) reacting the 2-D or 3-D DNA structures with a single strand specific nuclease to create cuts at the points of single strand sequence mismatches thereby removing the sequence error in the target polynucleotide sequence; (g) reacting the 2-D or 3-D DNA structures containing cuts at the points of single strand sequence mismatches with a polymerase to introduce the desired sequence at the cut point; and (h) optionally, releasing the target polynucleotide comprised of the desired sequence from the 2-D or 3-D DNA structures.

The single strand specific nuclease may be any suitable nuclease known in the art or may be chosen from Mung Bean endonuclease, T7 endonuclease I, E. coli endonuclease V, CEL endonuclease, S1 endonuclease, or P1 endonuclease. The polymerase may be any suitable polymerase known in the art or may be a polymerase with specific 3′-5′ and/or 5′-3′ proofreading functions. The polymerase may be E. coli DNA polymerase I (Pol I). The 2-D or 3-D DNA structures may contain one or more than one copy of the target polynucleotide and may be single stranded, double stranded or have a combination of single and double stranded elements. The 2-D or 3-D DNA structures may be of any 2-D or 3-D shape and may be a scaffold structure. The 2-D of 3-D DNA structures may be between 1 million Daltons and 10 million Daltons in molecular weight, between 0.5 million Daltons and 5 million Daltons in molecular weights, over 10 million Daltons in molecular weight, or under 0.5 million Daltons in molecular weight. The target polynucleotide may be one or more expression cassettes. The expression cassette may encode any therapeutic protein, including without limitation one or more proteins, antigens, antibodies, chimeric antigen receptors or T cell receptors. The expression cassette may also be excised from the 2-D or 3-D DNA structure in single stranded form and utilized to make recombinant viral vectors, including without limitation, rAAV. ssDNA obtained from the methods disclosed herein may be used for all or part of the rAAV manufacturing process and may be used to manufacture error mitigated AAV Rep and Cap genes, AAV helper genes, and/or the transgenes/cargo fragment. In an embodiment, a ligase enzyme may be used in lieu of or in conjunction with a polymerase to introduce the correct polynucleotide sequence at the point of the single stranded cut.

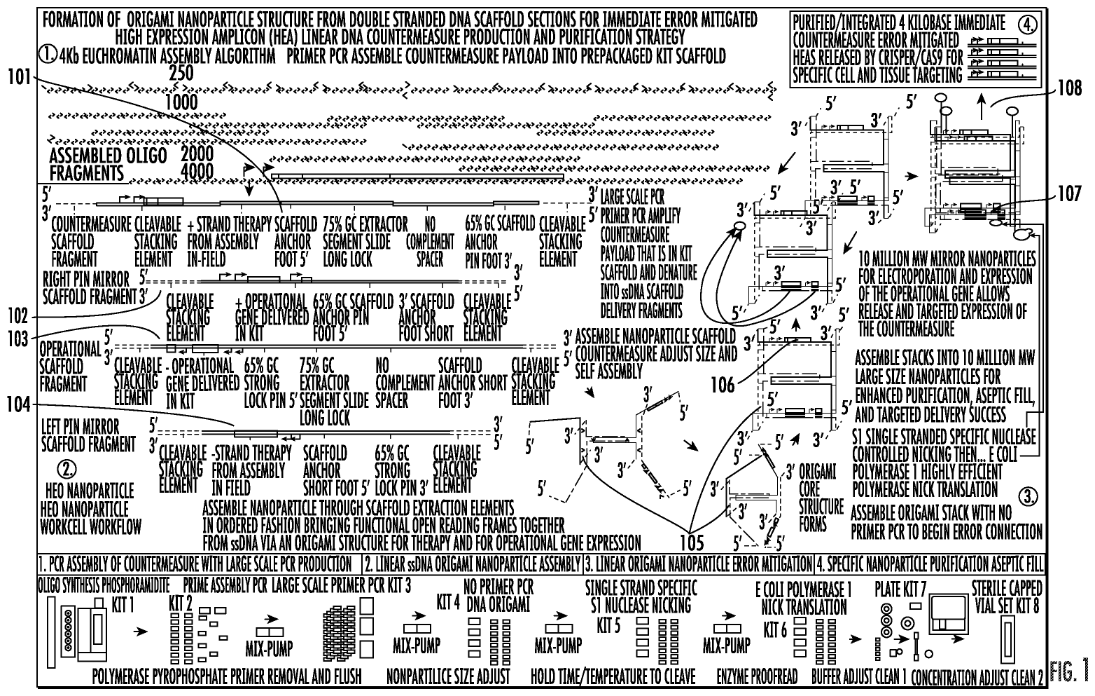

In another aspect, as shown in FIG. 1, the invention provides for a method of manufacturing a plurality of error mitigated polynucleotides of a desired sequence, said method comprising: (a) an expression vector scaffold polynucleotide comprising an expression cassette of a desired sequence assembled via seaming PCR to a polynucleotide that includes a 5′ cleavable stacking element, scaffold anchor, 75% GC extractor segment, a no-complement spacer, at least a 65% GC scaffold anchor, and a 3′ cleavable stacking element (101); (b) a right pin mirror scaffold polynucleotide comprising a 5′ cleavable stacking element, a negative strand (5′ to 3′) operational gene (encoding CRISPR/CAS9, Guide RNA, or DNA delivery components), at least a 65% GC scaffold anchor pin foot 5′, a 3′ scaffold anchor short foot, and a 3′ cleavable stacking element (102); (c) an operational scaffold polynucleotide comprising of a 5′ cleavable stacking element, a positive strand (5′ to 3′) operational gene (encoding CRISPR/CAS9, Guide RNA, or DNA delivery components) gene, at least a 65% GC strong lock pin foot 5′, at least a 75% GC extractor segment slide long lock, a no-complement spacer, a 3′ scaffold anchor short foot, and a 3′ cleavable stacking element (103); (d) a left pin mirror scaffold polynucleotide comprising an expression vector of a desired sequence assembled to a polynucleotide fragment which includes a 5′ cleavable stacking element, a positive strand (5′ to 3′) operational gene (encoding CRISPR/CAS9, Guide RNA, or DNA delivery components), scaffold anchor short foot 5′, at least a 65% GC strong lock pin 3′, and a 3′ cleavable stacking element (104), wherein the expression vector scaffold polynucleotide, right pin mirror scaffold polynucleotide, operational scaffold polynucleotide, and left pin mirror scaffold polynucleotide are denatured and annealed in a common reaction vessel to form self-assembling 2-D or 3-D DNA structures (105), wherein said 2-D or 3-D DNA structures provide for areas of single stranded sequence mismatches at the point in the expression cassette where the nucleotide sequence of the expression cassette differs from the desired sequence of the expression cassette (106); (e) Reacting the self-assembled 2-D or 3-D DNA structures with a single strand specific nuclease to create cuts at the points of single strand sequence mismatches in the expression cassettes thereby removing the sequence error in the expression cassette (107); and (f) reacting the self-assembled 2-D or 3-D DNA structures containing cuts at the points of single strand sequence mismatches to a polymerase to introduce the desired sequence at the cut point (107). Optionally, the error checked/corrected expression cassette is excised from the self-assembled 2-D or 3-D DNA structures (108).

The single strand specific nuclease may be any suitable nuclease known in the art or may be chosen from Mung Bean endonuclease, T7 endonuclease I, E. coli endonuclease V, CEL endonuclease, S1 endonuclease or P1 endonuclease. The polymerase may be any suitable polymerase known in the art or may be a polymerase with specific 3′-5′ and/or 5′-3′ proofreading functions. The polymerase may be E. coli DNA polymerase I (Pol I). The 2-D or 3-D DNA structures may contain one or more than one copy of the expression cassette and may be single stranded, double stranded or have a combination of single and double stranded elements. The 2-D or 3-D DNA structures may be of any 2-D or 3-D shape and may be a scaffold structure. The 2-D of 3-D DNA structures may be between 1 million Daltons and 10 million Daltons in molecular weight, between 0.5 million Daltons and 5 million Daltons in molecular weight, over 10 million Daltons in molecular weight, between 20 million Daltons and 100 million Daltons in molecular weight, or under 0.5 million Daltons in molecular weight. In an embodiment, a ligase enzyme may be used in lieu of or in conjunction with a polymerase to introduce the correct polynucleotide sequence at the point of the single stranded cut.

The 2-D or 3-D DNA structures may be formed by DNA self-assembly. The 2-D or 3-D DNA structures may be derived from the denaturing of several double stranded DNA constructs that, once denatured, are comprised of ssDNA that are complimentary or partially complementary in sequence to other ssDNA constructs present in the reaction vessel or introduced into the reaction vessel. The ratio of the ssDNA elements may be 1 to 1 or may be other than 1 to 1. Upon annealing, the complementary or partially complementary ssDNA constructs self-assemble into 2-D or 3-D structures that contain the target polynucleotide. Exemplary 2-D or 3-D double stranded DNA structures include, without limitation, DNA scaffolds, DNA tiles, DNA lattices, DNA cross tile lattices, DNA hexagonal lattices or any other shape or nanostructure. The 2-D or 3-D DNA structures may be designed with the aid of computer software and may be dictated by the specific sequence of the target polynucleotide. The 2-D or 3-D DNA structures may be anchored to the reaction vessel, an anchor point, or other substrate such that the DNA structure remains in a fixed position after assembly. The 2-D or 3-D DNA structures may also be removably affixed to the reaction vessel, an anchor point, or other substrate. The 2-D or 3-D DNA structures may remain in a fixed position for the duration of being reacted with a single strand specific nuclease and polymerase or ligase to remove sequence error from the target polynucleotide. Self-assembly of the 2-D or 3-D DNA structures may be completed in 1 or more thermocycles, alternating between denaturing and annealing temperatures.

The self-assembled 2-D or 3-D DNA structures may be double stranded and may form a scaffold. Two basic principles may be employed in the design of such scaffold folding paths—folding path asymmetry and periodic convergence of the two ssDNA scaffold strands. Asymmetry in the folding path minimizes unwanted complementarity between staple sequences. In addition, incorporating an offset between the folding paths of each ssDNA scaffold strand reduces the number of times that complementary portions of the strands are brought into proximity with one another, both of which decrease the likelihood of dsDNA scaffold assembly. Meanwhile, the folding paths of the two ssDNA scaffold strands are designed to periodically converge to promote the assembly of a single, unified structure rather than two individual ones. This methodology can be utilized to reliably assemble dsDNA scaffolds or other 2-D or 3-D nanoparticles from dsDNA.

The 2-D or 3-D DNA structures may contain one expression cassette, or the 2-D or 3-D DNA structures may contain more than one expression cassette. The more than one expression cassettes in a 2-D or 3-D DNA structures may be of identical sequence or they may be of different sequences. The expression cassettes may encode any therapeutic protein, including without limitation one or more proteins, antigens, antibodies, chimeric antigen receptors, T cell receptors or any combination thereof or other combinations of therapeutic payloads. The expression cassettes may be also be used to create ssDNA for viral vector production or recombinant viral vector production.

The target polynucleotide may be excised from the self-assembled 2-D or 3-D DNA structures via any sequence specific methodology known in the art. Exemplary methods include the use of a restriction site, a restriction enzyme, CRISPR/Cas9, Cas9, or other restriction endonuclease. The target polynucleotide may also remain within the self-assembled 2-D or 3-D DNA structures and be administered to a subject as part of the 2-D or 3-D DNA structure nanoparticle. The delivered nanoparticle may contain one or more target polynucleotides engineered to express multiple therapeutic proteins.

### System for Manufacturing a Plurality of Error Mitigated Polynucleotides of a Desired Sequence

In another aspect, the invention provides for a system for manufacturing a plurality of error mitigated polynucleotides of a desired sequence, said system comprising: (a) a thermocycler; (b) a liquid handler; (c) microfluidic pumps; and (d) DNA purification systems. All components may be in fluid communication.

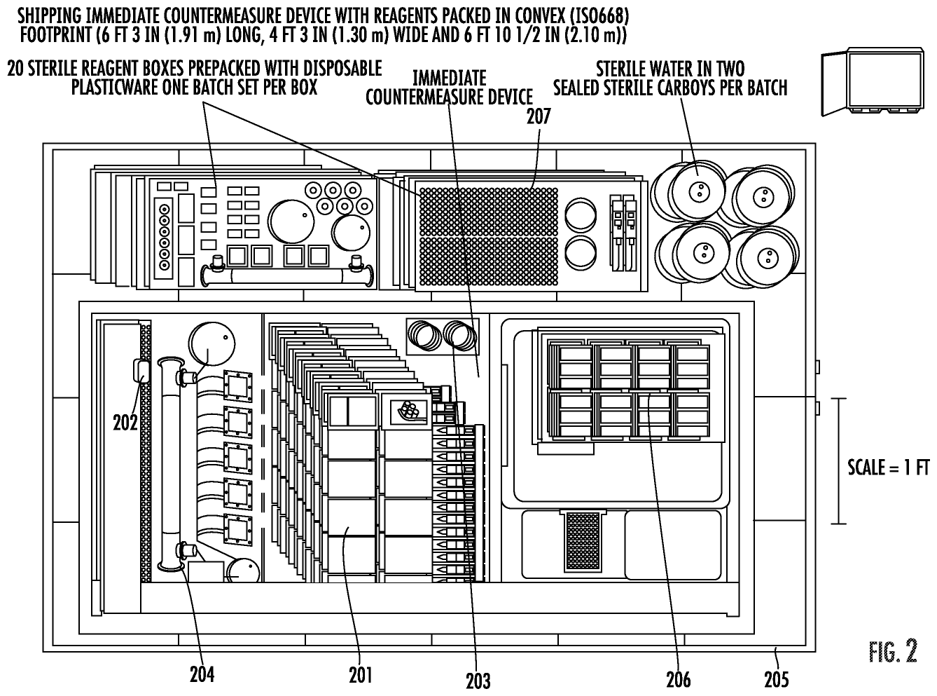

Turning to FIG. 2, the system may be comprised of one or more thermocyclers (201), a liquid handler (202), microfluidic pumps (203) and one or more DNA purification systems (204). The system may be entirely contained in closed unit (205) or the system may disposed in or more units or locations. The closed unit maybe a CONEX container. The system may also contain a next generation sequencing device (206) and/or a polynucleotide assembly device (207). The system may optionally also contain an aseptic fill apparatus. The system may be configured to perform the methods of manufacturing a plurality of error mitigated polynucleotides of a desired sequence disclosed herein.

The thermocycler (201) may be any thermocycler currently known in the art. The thermocycler may be configured to undertake large-scale PCR. The thermocycler may be one or more microfluidic devices or one or more “lab on a chip” systems. One or more thermocyclers may be used in a parallel or massively parallel system and may be comprised of a plurality of microfluidic devices configured for thermocycling. The microfluidic devices may be configured to operate as a plurality of separate reaction vessels or may be configured to run in a continuous flow modality. A thermocycler configured to undertake large-scale PCR may also be configured to run in a continuous flow modality.

The polynucleotide assembly device (207) may be any polynucleotide assembly device known in the art. The polynucleotide assembly device may utilize artificial gene synthesis that may include photolithography, oligonucleotide synthesis, solid-phase DNA synthesis or any other means of gene synthesis know in the art capable of producing polynucleotides of the necessary length and fidelity. The polynucleotide assembly device may also utilize the process of oligonucleotide assembly to form larger polynucleotides. An exemplary oligonucleotide assembly device is the BioXp™ (SGI-DNA, United States). The polynucleotide assembly device may also utilize seeming PCR to assemble polynucleotides from oligonucleotides.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

What is claimed is:

1. A method of manufacturing a plurality of error mitigated target polynucleotides of a desired sequence, said method comprising:
Obtaining a quantity of target polynucleotides of a desired sequence;
Amplifying the target polynucleotides via the polymerase chain reaction (PCR) to create a first set of double stranded amplicons;
Denaturing the first set of double stranded amplicons;
Annealing the denatured amplicons to create a second set of double stranded amplicons that contain areas of single stranded sequence mismatches at the point in the amplicons where the nucleotide sequence differs from the desired sequence;
Reacting the second set of double stranded amplicons with a single strand specific nuclease to create cuts at the points of single strand sequence mismatches in the second set of double stranded amplicons to remove sequence error; and
Reacting the second set of double stranded amplicons containing cuts at the points of single strand sequence mismatches with a polymerase to introduce the desired sequence at the cut point.

2. The method of claim 1, wherein the single strand specific nuclease is chosen from the group consisting of Mung Bean endonuclease, T7 endonuclease I, E. coli endonuclease V, CEL endonuclease, S1 endonuclease and P1 endonuclease.

3. The method of claim 1, wherein the polymerase is E. coli DNA polymerase I (Pol I).

4. The method of claim 1, wherein the polymerase is any polymerase with 3′-5′ and/or 5′-3′ proofreading functionality.

5. The method of claim 1, wherein the polynucleotides of a desired sequence contains an expression cassette and noncoding nucleotides with a G-C content of at least 65%.

6. The method of claim 1, wherein the target polynucleotides are an expression cassette.

7. The method of claim 6, wherein the target polynucleotides are an expression cassette expressing a desired antigen.

8. A method of manufacturing a plurality of error mitigated polynucleotides of a desired sequence, said method comprising:
Obtaining a quantity of target polynucleotides of a desired sequence;
Amplifying the target polynucleotide via the polymerase chain reaction (PCR) to create a first set of double stranded amplicons;
Assembling the first set of double stranded amplicons with a DNA scaffold via seaming PCR to create a second set of double stranded amplicons comprising the target polynucleotide and scaffold DNA;
Denaturing the second set of double stranded amplicons;
Reacting the denatured second set of double stranded DNA amplicons with one or more complementary or partially complementary DNA sequences to form 2-D or 3-D DNA structures wherein the complimentary target polynucleotide sequences are forced to hybridize in the 2-D or 3-D DNA structure to create areas of single stranded sequence mismatches at the point in the target polynucleotide sequence where the sequence differs from the desired sequence;
Reacting the 2-D or 3-D DNA structures with a single strand specific nuclease to create cuts at the points of single strand sequence mismatches thereby removing the sequence error in the target polynucleotide sequence;
Reacting the 2-D or 3-D DNA structures containing cuts at the points of single strand sequence mismatches with a polymerase to introduce the desired sequence at the cut point; and
Releasing the target polynucleotide comprised of the desired sequence from the 2-D or 3-D DNA structures.

9. The method of claim 8, wherein the 2-D or 3-D DNA structures contain more than one copy of the target polynucleotide.

10. The method of claim 8, wherein the target polynucleotide is an expression cassette.

11. The method of claim 8, wherein the 2-D of 3-D DNA structures are double stranded.

12. The method of claim 8, wherein the 2-D of 3-D DNA structures are a scaffold shape.

13. The method of claim 8, wherein the 2-D of 3-D DNA structures are between 1 million Daltons and 10 million Daltons in molecular weight.

14. The method of claim 8, wherein the released target polynucleotide comprised of the desired sequence is used as a template for RNA.

15. A method for producing an antigen specific immune response in a subject, said method said method comprising the steps of:
Choosing one or more desired antigens for expression within a subject;
Obtaining a quantity of target polynucleotides of a desired sequence, said target polynucleotides of a desired sequence containing one or more expression cassettes for the desired one or more antigens;
Amplifying the target polynucleotides via the polymerase chain reaction to create a first set of double stranded amplicons;
Denaturing the first set of double stranded amplicons;
Annealing the denatured amplicons to create a second set of double stranded amplicons that contain areas of single stranded sequence mismatches at the point in the amplicon where the nucleotide sequence differs from the desired sequence
Reacting the second set of double stranded amplicons with a single strand specific nuclease to create cuts at the points of single strand sequence mismatches in the second set of double stranded amplicons to remove sequence error
Reacting the second set of double stranded amplicons containing cuts at the points of single strand sequence mismatches with a polymerase to introduce the desired sequence at the cut point thereby create a plurality of double stranded amplicons with the desired sequence containing expression cassettes for the desired one or more antigens;
Formulating the plurality of double stranded amplicons with the desired sequence containing expression cassettes for the desired one or more antigens into a therapeutic dose; and
Administering the therapeutic dose to a subject, wherein the subject produces the desired one or more antigens in response to the therapeutic dose.

16. The method of claim 15, wherein the plurality of double stranded amplicons with the desired sequence containing expression cassettes for the desired one or more antigens is formulated with lipid nanoparticles to create a therapeutic dose.

17. The method of 16, wherein the therapeutic dose is administered via injection.

18. The method of claim 15, wherein the single strand specific nuclease is chosen from the group consisting of Mung Bean endonuclease, T7 endonuclease I, E. coli endonuclease V, CEL endonuclease, S1 endonuclease and P1 endonuclease.

19. The method of claim 15, wherein the polymerase is E. coli DNA polymerase I (Pol I).

20. The method of claim 15, wherein the polymerase is any polymerase with 3′-5′ and/or 5′-3′ proofreading functionality.
